(19) United States
(12) Patent Application Publication
(10) Pub. No.: US 2010/0030059 A1
(43) Pub. Date: Feb. 4, 2010

(54) Method for Measuring Information of Biological Systems

(75) Inventor: Ralf Otte, Weinheim (DE)

Correspondence Address: SHOEMAKER AND MATTARE, LTD, 10 POST OFFICE ROAD - SUITE 100, SILVER SPRING, MD, 20910, US

(73) Assignee: TECDATA AG, Uzwil (CH)

(21) Appl. No.: 12/527,358

(22) PCT Filed: Feb. 14, 2008
(86) PCT No.: PCT/CH08/00061

§ 371 Date: Aug. 14, 2009

(30) Foreign Application Priority Data
Feb. 15, 2007 (DE) 102007008021.4

(51) Int. Cl. A61B 5/05; A61B 5/00; G01V 9/00

(52) U.S. Cl. 600/407; 600/300; 324/800

(57) ABSTRACT

The invention relates to a method for measuring information of biological systems. The aim of the invention is to receive signals using less energy. To achieve this, random generators are used as receivers (B) of low-energy quanta, since the random generators can be regarded and implemented as antennae and receivers of signals of this type. The extensive natural transmission range of low-energy quanta can also be used to receive information from spatially remote systems.

The invention relates to a method for measuring information of biological systems.

The method is suitable for measuring the entropy state and information state of a biological system.

It is generally known for information to be measured, to be transmitted, to be received and to be evaluated by suitable methods.¹ ¹ Fritsche, Witzschel: Informationsubertragung [Information transmission], VEB Verlag Technik, Berlin, 1989

One disadvantage of the conventional methods is that a relatively large amount of energy must be consumed in order to transmit information. Even the most modern types of mobile telephones, for example, consume several watts or milliwatts of transmission power in order to transmit speech information.

In order to transmit the information (messages) by means of electromagnetic waves, the messages are modulated onto a carrier wave at a suitable frequency and with a suitable power (for example amplitude or frequency modulation), and are sent, and this modulated carrier wave can then be received, decoded and processed further by a receiver. In this case, antennas of suitable length (λ/2 or λ/4 dipoles) or other resonators with a suitable characteristic impedance or radiation impedance may be used as receivers for electromagnetic waves. It is prior art to receive or to transmit waves at a frequency of, for example, 30 kHz to 30 THz, corresponding to wavelengths from 10 km to 10 μm. Waves at higher frequencies, for example infrared or optical frequencies, are also technically processed and, furthermore, in a number of physical specific disciplines (for example nuclear physics), electromagnetic waves are used at an extremely high frequency and with extremely high energy, for example gamma rays.

However, it is problematic or in some cases impossible to receive, to process and to send very long electromagnetic waves, that is to say waves whose frequency is in the extremely low range, for example in the Hertz range, and which therefore have wavelengths of several hundred or thousand kilometers. This is therefore technically difficult because resonators (tuned circuits) with an extremely low resonant frequency and nevertheless a suitable characteristic impedance are required for reception, and this necessitates antenna installations with a very large physical extent. Technical approaches exist for use of the Earth's ionosphere itself as an antenna, and therefore for producing or manipulating waves with a very long wavelength, although this requires a very high degree of hardware complexity and is therefore feasible for only a small number of facilities. However, even these approaches fail when one wishes to receive electromagnetic waves with wavelengths of several tens of thousands of kilometers.

It is also known that the waves have both a particle and a wave characteristic, and that the associated characteristics can be determined using different measurement methods. It is state of the art that electromagnetic waves comprise quanta which obey the laws of quantum physics. One example is the known double-slot experiment, which indicates the wave character of such photons or quanta, and other experiments which, for example, measure the radiation pressure, indicate the particle character of such quanta². ² D. I. Blochinzew: Grundlagen der Quantenmechanik [Principles of quantum mechanics], Verlag Harri Deutsch, Frankfurt, 1988

Since there is a unique mathematical relationship between frequency and energy, it is impossible according to the present prior art to receive and to deliberately transmit quanta, for example electromagnetic quanta, with extremely low energy (at an extremely low frequency).

The invention is based on the object of specifying a method and a device by means of which quanta, so-called low-energy or very low-energy quanta, that is to say for example quanta with energies of less than 10⁻³² Joules—can be measured and received in order in this way to provide novel application possibilities for information transmission from biological to technical systems.

This object is achieved by a method specified in claim 1 and by a device specified in claim 15 for measuring information from technical or biological systems, in which the low-energy signals are received and evaluated by suitable receivers, so-called random number generators, with the physical relationship between frequency and energy according to E=h*f being used (where E is the energy of one quantum, f is its frequency and h=6.626*10⁻³⁴ Js, the so-called Planck's constant³), in order to determine the energy of the signal to be received, and to design the random-number generators as receivers or transmitters of such low-energy signals. ³ Brandt, Dahmen: Quantenmechanik auf dem Personalcomputer [Quantum mechanics on a personal computer], Springer-Verlag, Berlin, 1993

Advantageous refinements are disclosed in the dependent claims.

The new approach for measurement of very low energies and therefore very low frequencies results in technical application options which have hitherto been unknown.

In order to assist understanding of the invention, an information conservation rule of nature is postulated in parallel with the energy conservation rule, which states that information can never be lost. Like energy as well, information can only be converted from one form (for example random information≡entropy) to a different form (structure information), i.e.

total information I=structure information S+random information H+remaining information U

I=S+H+U (1.1)

U represents an unknown type of information which may also need to be introduced. At the instant at which a random information item H is converted to structure information S by semantic knowledge, nothing has changed in the overall information I of an object, in accordance with equation (1.1).

The abovementioned parallels between energy conservation and information conservation mean that an entropy exchange (information exchange) must take place between two objects of different entropy density (information density), in the same way that an energy exchange takes place between two objects of different energy, until the energy difference is equalized.

If there is an entropy difference ΔH=H₁−H₂ between two objects 1 and 2 and there is a capability, of whatever type, to equalize this, then the entropy flow H_{F} is:

H_{F}˜ΔH (1.2)

The entropy flow HF is in this case proportional to the entropy gradient of the two objects, and its direction is such that the entropy flows from the object of high entropy (for example H₁) to the object of low entropy (for example H₂) until the entropy has been equalized.

As a result of the relationship (1.1) between entropy H and information I, the entropy transfer can be equated to an information transfer, that is to say information transfer and entropy transfer are regarded as equivalent in the description, since they can mathematically be converted to one another. For example, the total information in a bit sequence of 20 bits is 20 bits. How many bits thereof are structure information and how many are random information in this case depends on the context, but the two can be converted to one another. However, for simplicity, the following text refers to entropy transfer.

It is known that information is exchanged between two objects by means of so-called quanta (for example quanta of the electromagnetic field, that is to say photons) of a specific energy and at a specific frequency. In this case, it is in general normal for quanta with a specific energy, which are emitted as an electromagnetic wave at the wavelength λ to be received by specific apparatuses and methods. Tuned circuits such as those in any radio receiver are normally used for this purpose. The tuned circuit must in this case be tuned to the frequency f of the wave (where f=λ/c and c is the speed of light), and an antenna is required for reception. It is known that, inter alia, the antenna must obey the λ/4 law, that is to say the length of the antenna dipole should be λ, λ/2 or λ/4⁴. ⁴ Liebscher: Rundfunk-, Fernseh-, Tonspeichertechnik [Broadcast radio, television, audio storage technology], VEB Verlag Technik, Berlin, 1981

It is also known that these methods and devices can receive only waves up to a specific wavelength, for example long waves. Waves whose wavelength is even longer (for example 10 000 km or more) and which are therefore at an extremely low frequency with a low energy level, cannot be received according to the present prior art.

By way of example, conventional television waves are at a frequency of more than 30 MHz, that is to say wavelengths of less than 10 meters. Conventional LW radio waves are at a frequency of >30 kHz, that is to say wavelengths of less than 10 kilometers. The electromagnetic radio waves and frequencies of conventional technical applications normally vary within this range. However, there are numerous technical applications using much higher frequencies, for example microwaves (λ=1 mm to 1 m, f=300 MHz to 300 GHz), spectroscopes (λ=30 μm to 3 mm, f=0.1 THz to 10 THz) or infrared remote controls (λ=780 nm to 1 mm, f>300 GHz). Very long waves, such as those which are received and/or transmitted by specific installations are, for example, at a frequency of 3 kHz and their wavelength is therefore <100 km. The reception of waves (quanta) at a wavelength of several hundred or thousand kilometers is at present technically impossible, or is possible only with an extremely high degree of complexity.

The object of the invention is to develop a method for measuring information of biological systems, which allows waves at extremely long wavelengths (up to several thousand kilometers and more) and therefore with extremely low energy, to be received.

The human brain oscillates, inter alia, at frequencies around 8 Hz (delta range: 0.5 to 4 Hz, theta frequencies: 4 to 7 Hz, alpha range: 7 to 13 Hz, beta range: 13 to 32 Hz, gamma range up to 40 Hz), and this is used by numerous EEG applications. In this case, the oscillation is detected by electrodes on the surface of the head, and is evaluated. The oscillations in the brain therefore also influence its surrounding area, and this is a fundamental precondition of EEG output. The range to which this influence extends and whether these oscillations are also emitted as electromagnetic waves are at present unknown. It is assumed that the brain oscillations form corresponding electromagnetic quanta around the generator source. By way of example, 8 Hz oscillations produce electromagnetic 8 Hz quanta. Whether these quanta are also actually emitted is not relevant to the invention since waves are detected only in the so-called far field of antennas (see page 8).

According to the generally known equations λ=c/f and E=h*f, and using h≈6.63*10⁻³⁴ J, the wavelength following 8 Hz, for example, and therefore the energy of the 8 Hz quanta that follow this correspond to: λ=37 500 km and E=5.3*10⁻³³ J.

From the Heisenberg uncertainty theorem⁵ ⁵W. Heisenberg: “Über den anschaulichen Inhalt der quantentheoretischen Kinematik und Mechanik” [On the evident content of quantum-theoretical kinematics and mechanics] 1927, in “Dokumente der Naturwissenschaft” [Documents of Natural Science], Physics, Battenberg Verlag, Stuttgart, 1963

Δp*Δx≧h (2.1.)

where Δp is the accuracy of the impulse, Δx is the accuracy of the location, and h is Planck's constant, it is evident that these 8 Hz quanta are undefined over the location of 37 500 km, which possibly allows the brain to receive information from a long distance away from other signal sources directly and at the speed of light, if it is made able to “filter out” the quanta with this low energy (E=5.3*10⁻³³ J) from the other energies in the brain.

The following terms are introduced for the rest of the description (the subdivision is simplified and serves only to clarify the terminology, the physically exact limits can be found in the literature):

| | | Wavelength | Energy | | |
|---|---|---|---|---|---|
| | Frequency | examples | examples | Name | Abbreviation |
| 1 | >10²⁰ Hz | <3 pm | 6.63 * 10⁻¹⁴ J | Gamma quanta | GQ |
| 2 | 10¹⁴ Hz | 400-700 nm | 2.8 * 10⁻¹⁹ J-4.9 * 10⁻²⁴ J | Photon | — |
| | | | | quanta/photons | |
| 3 | 100 kHz-5 GHz | 6 cm-3 km | 6.63 * 10⁻²⁹ J-3.31 * 10⁻²⁴ J | Radio quanta | RQ |
| 4 | 1 Hz-100 Hz | 3000 km-300 000 km | 6.63 * 10⁻³⁴ J-6.63 * 10⁻³² J | Low-energy quanta | LEQ |
| 5 | <1 Hz | >300 000 km | 6.63 * 10⁻³⁴ J | Very low-energy | LSTEQ |
| | <<1 Hz | | (e.g. also | quanta = entropy | |
| | | | 10⁻¹⁰⁰ J) | quanta | |

A further object of the invention is to provide a device for measuring information of biological systems.

The invention makes it possible to receive LEQ quanta or LSTEQ quanta, with it also being possible to receive other quanta (for example radio quanta). Nowadays, suitable technical solutions (radio, television and mobile-telephone receivers) exist for receiving radio quanta, but no receivers yet exist for receiving low-energy quanta, for which reason the description concentrates on the latter. The technical embodiment for receiving both low-energy quanta (4, 5) is the same, with the only difference being the application options. For example, LEQ quanta are suitable for remote monitoring or diagnosis, and LSTEQ quanta are predestined for prediction tasks. The terms low-energy quanta and very low-energy quanta are, however, always used synonymously in the following text where no distinction is necessary.

There are a number of possible ways to carry out the invention, two of which will be mentioned by way of example, with the variant 2.1.b) being described in more detail:
2.1.a) Reception of the signals by receivers whose conductor tracks have been appropriately physically designed and manufactured. By way of example, even in 1985, the conductor track lengths on integrated circuits were around 40 km long. If it is assumed that these conductor tracks correspond to technical antennas, it was therefore possible to receive frequencies of 7.494 kHz.
According to the invention, appropriate receivers which have a specific conductor track configuration are constructed for receiving signals with very low energy. This embodiment is admittedly technically demanding, but is physically and conceptually trivial.
One interesting side-effect is that, even today, all technical devices with conductor track runs such as these, for example computer processes, deliberately or inadvertently receive and transmit such signals with very low energy, and these cannot be screened without a clearing system (see below). Communication therefore takes place deliberately or inadvertently all the time between, for example, processors and other processors or biological systems.
A partial objective of the invention is therefore to specify a method and a device, a clearing system, which restricts or impedes the transmission and therefore external reception of information that is worth protection.
2.1.b) Reception of signals by measuring the influence of microsystems, such as atoms, electrons, etc. Beyond a certain very low energy level, the complexity of the design and construction of antennas according to engineers is no longer possible or is too expensive, which means that a fundamentally different method must be used. According to the invention, systems are used for this purpose, for example, which have a certain arrangement of microparticles, whose change can be recorded.
By way of example, boundary surfaces of semiconductors, non-reactive resistors, radioactive decomposition processes, designs in which photons are reflected with a certain probability, and many more, are suitable for this purpose.
Changes in microparticles, for example by their impulse or their spin being changed, can be measured by suitable devices. Specific magnetic measurement devices, so-called “spin measurement devices” can be used for measuring spin changes, as is already done nowadays, in a rudimentary form, in magnetic resonance imaging.

A novel measurement method, based on 2.1.b), for measuring quanta with very low energies is represented by the use of noise generators, such as those which are conventionally used for generating random numbers.

According to the invention, a random process is therefore used for receiving signals (quanta). The random process must be suitably designed for receiving signals of very low energy (LEQ, LSTEQ quanta).

Suitable random processes can be implemented by mathematical random number generators (pseudorandom number generators, time decomposition generators, n random number generators) or physical random number generators (physical noise generators). The noise signals of physical noise generators may in this case be created by a very wide range of physical processes, for example by thermal noise, radioactive noise, magnetic noise, otoacoustic noise, biological noise, photon noise etc. In these processes, the movement of microparticles (for example electrons at semiconductor boundary surfaces in the case of thermal noise) or photon quanta in the case of photon noise (Quantis devices⁶) are converted to an electrically measurable signal, which is then interpreted as a noise signal (random signal). ⁶ www.idquantique.com

According to the invention, signals from random processes are often not actual random signals but they indicate the reception of very low-energy waves whose energy is just sufficient, for example, to influence the microparticles (electrons) of a thermal noise generator.

One known example for the reception of broadband signals is provided by so-called fractal antennas which are used nowadays in numerous applications (for example mobile telephones, cars), since it is possible, by miniaturization, to produce extremely small antennas which can receive the desired wavelengths (Fractal Antennas: A Novel Antenna Miniaturization Technique and Applications, J. Gianvittorio and Y. Rahmat-Samii in IE-EE Antennas and Propagation Magazine Vol. 44, No. 1, February 2002).

According to the invention, however, antennas such as these are also formed on the boundary layers of the pn junctions of semiconductors since the doping process creates molecule structures which are similar to technically produced fractal antennas, although on a different scale. The naturally formed fractal antennas of semiconductor components are therefore suitable for reception of broadband signals. Since their structures—although folded—are physically large, they are also suitable for receiving low-frequency signals. This means that even simple diodes can be used to receive LEQ and LSTEQ quanta.

Avalanche diodes are particularly suitable for receiving biological signals and zener diodes are suitable for receiving technical signals. Alternatively, the conductor tracks of complex digital switching networks, such as processors, are also technically suitable for receiving the above-mentioned LEQ and LSTEQ quanta.

The microparticles and their natural or technical use for tuned circuits are therefore, according to the invention, antennas for LEQ and LSTEQ quanta. Their three-dimensional arrangement on a boundary surface governs the capability to receive signals at a specific wavelength, since the antennas and the wavelength of the signal must satisfy a specific resonance condition. The length of an antenna such as this on semiconductor boundary surfaces may be several meters to thousands of kilometers, thus allowing reception of signals at a corresponding wavelength.

It is generally known that the semiconductor effect is a quantum-mechanical effect since, as a result of entanglement of electrons (holes), entire columns of electrons (holes) act like a single electron (hole), and can migrate through the semiconductor. Thus, in the end, reception by means of semiconductor noise generators is based on a quantum-mechanical process (Robert B. Laughlin, Abschied von der Weltformel [Departure from the world formula], Piper Verlag, Munich, 2007). This is advantageous since this makes it possible to deliberately make use of quantum-mechanical effects. Every semiconductor is an information receiver based on a quantum-mechanical process, which obeys the laws of emergence.

The physical effects of self interference of quanta as described in this invention are made technically useful by the use according to the invention in particular of semiconductors as antennas for very long waves, that is to say low-energy quanta (LEQ, LSTEQ). Semiconductor-based noise generators are therefore information receivers which, based on physics, allow quantum effects of the low-energy range in applications which can be used technically. From the technical point of view, it is therefore irrelevant whether the quanta are received by fractal antennas on the boundary surfaces of the semiconductors (and therefore satisfy the known λ/4 conditions, page 5) or whether their reception is made possible by time self-entanglement of the quanta, and therefore are created directly by time sampling of the random signal.

Specific patterns of emergence are created in physical and/or time proximity. It is known from the phase transitions theory that, in the case of a spatial process, new patterns can be created suddenly, affecting all the particles in the vicinity. This is based, for example, on the characteristics of the metallic phase, the sudden freezing of water, and much more. In precisely the same way, patterns can also be created by emergence when the processes are close in time. In this case, the distance is irrelevant. These characteristics of time emergence can be used deliberately since this allows entropy to be transported (even over relatively long) distances. The microparticles of the transmitter and receiver in suitable circumstances can produce patterns that are synchronous in time.

Random number generators and noise generators are therefore, according to the invention, information receivers and entropy receivers. By way of example, they are therefore suitable for use as entropy measurement devices for the surrounding area if, for example, one wishes to identify fault states. Random number generators permanently receive the energy and entropy (information) from the objects surrounding them.

FIG. 1 shows a device DEVICE for receiving quanta. The quanta EQ from the environment device BIO at a distance s from the appliance DEVICE are received by a random number generator RNG and change its noise response. The resultant random number sequences⁷ are passed to a processing unit PRZ, where they are evaluated and compared. ⁷ Although the random number sequences of a noise generator are, according to the invention, created by the reception of quanta, that is to say they are causal, they will nevertheless be referred to in the following text as random number sequences because these sequences pass all the statistical tests for randomness. This is because of the fact that the tests carry out a statistical analysis of the sequence and not a semantic analysis. A semantic evaluation has also not been necessary until now since the sequences from noise generators have actually and not just apparently been assumed to be random. Although there is a causal influence of random number generators, their sequences always appear random since the generators represent an additive and/or multiplicative superimposition of a very large number of complex states of received quanta.

If objects with high entropy are located in the vicinity of noise generators, then these objects emit the entropy and the noise generator receives the emitted entropy, and this could be identified, for example, from the fact that the entropy of the noise generator rises, that is to say the fluctuation of the numerical sequences generated by the noise generator increases. Entropy exchange takes place between the environment ENV and the noise generator RNG. On the other hand, a noise generator can also emit entropy to the environment when a receiver is in resonance with it and an entropy gradient is present.

As in information technology, the resonance condition is normally satisfied precisely when the receiver can receive the frequency (wavelength). However, in contrast to conventional information technology, this always relates to the exchange of very low-energy quanta, that is to say quanta at a very low frequency and a very long wavelength. Other forms of the resonance condition are disclosed further below with respect to so-called calibration. Particularly when information is being exchanged, a semantic resonance condition must be created, since the receiver would otherwise not identify the information from the transmitter as such at all but would in fact interpret this as a random signal.

One example of random number generators being able to receive low-energy quanta (even LEQ quanta) is well known to those skilled in the art. For example, when designing random number generators (for example thermal noise generators), particular care is taken to ensure that these generators are screened from alternating-current influences. In Europe, the alternating current is at a frequency of 50 Hz, which, according to E=h*f, corresponds to its quanta having an energy of 3.31*10⁻³² J and being at a wavelength of approximately 5995 km. Random number generators can therefore already today receive quanta with an energy of 3.31*10⁻³² J. If the generator is not very well screened or is not designed by means of suitable measures such as the construction of balanced circuits for the alternating-current components in the noise to cancel one another out, then the influence of the alternating current in the trend image of a noise sequence indication system can even be identified with the naked eye. Random number generators that have been influenced in this way therefore do not pass statistical tests for randomness. The “non-voluntary” reception of low-energy quanta (for example 50 Hz quanta) in random number generators therefore nowadays has an extremely disruptive effect although, so far, this has not been identified per se.

One major component of an information exchange of low-energy quanta such as this is that, using methods that are already known, screening can be carried out only with difficulty since 1) the energy of the quanta is so low that the quanta can often interact only to a very minor extent with the surrounding materials (electrons, atoms, nuclei) and can therefore pass through these materials and 2) particularly in the case of low-energy quanta, effects of the electromagnetic near field, in particular the radial component effect (longitudinal component) are used. This means that myriads of quanta are permanently flooding our environment. Every biological and technical system needs to filter out and further process those quanta which are useful to it from this “quantum mixture”, simply by suitable filtering, addressing and calibration routines.

If the detectors are used to receive very low frequency signals, then this furthermore results in special factors. It is known from information technology theory that electromagnetic waves have two fundamentally different areas: the near field and the far field (Zinke, Brunswig, Hochfrequenztechnik 1 [Radio-frequency technology 1], Springer Verlag, 6th edition, Berlin, 2000). In the technically conventional case, the characteristics of the far field are used, and these are essentially based on the transversal characteristics of Hertzian waves. This is because the expression “near field” is used only up to a distance of one to two times the wavelength, and beyond this is referred to as a far field. The frequencies which are normally used nowadays therefore have a near field which is small, with a maximum of only a few centimeters or meters. This is not applicable to LEQ frequencies. The waves used here have a wavelength of up to 300 000 km (1 Hz) generally 30 000 km (10 Hz). For example, if f=50 Hz, near field still exists at a distance of 1000 km (ebenda, page 386). The characteristics of the near field must therefore also be taken into account for any application on the Earth. It is now also known from information technology that, particularly in the near field, every electromagnetic signal also has longitudinal components (radial components); this longitudinal component in particular contributes to the separation of the Hertzian wave (ebenda, page 388) In the near field, magnetic and electrical components of the field are phase-shifted through 90 degrees, but they are not in the far field. The near field of a Hertzian dipole is primarily of an electrical nature. Since the longitudinal components fall with 1/r³ (when r is the distance to the transmitter), the transversal components fall only at 1/r², however, only the transversal characteristics of the wave therefore still exist beyond a certain distance from the transmitter, and this is made useful by the normal technical applications nowadays. However, there are other phenomena in the near field. The longitudinal component can be screened only with difficulty using conventional methods. However, this means that the signal sources which, for example, oscillate in the 10 Hz range, build up a near field, which can be screened only with difficulty, from 10 000 to 30 000 km around themselves.

Low-energy quanta have a high degree of spatial penetration and can be received virtually everywhere on the surface of the Earth.

If one assumes that the human brain produces oscillations at 10 Hz, inter alia, then it is evident that a very strong electromagnetic near field exists around every brain, with a longitudinal component which can be received by suitable receivers even at long distances (radii up to 30 000 km).

In this case, it is irrelevant whether these quanta are also emitted, and it is sufficient for the invention that a quantum field is formed around every biological system, which physically corresponds to the near-field characteristics of antennas and which can be measured and evaluated using suitable means, by specially designed receivers.

Information can therefore be received about desired objects. Because of the near-field character of the low-energy quanta, the objects may be at a long physical distance, which may be several thousand kilometers or considerably more. The objects may be biological systems of any type, cells, organs, animals, bacteria, plants or parts thereof.

States of biological systems (for example brain states) can therefore be received by suitable receivers everywhere on the Earth. The signal transmission is therefore reduced to the reception and in particular filtering out the desired signals from the signal mixture at the receiver, because every semiconductor component receives the signals from millions of biological or technical transmitters, and these are all superimposed. The superimposition produces from this a random signal, which can be identified by a person skilled in the art and which actually satisfies all the criteria of a random signal (autocorrelation etc.).

The low-energy quanta can be transmitted over long distances in the near-field area. Nevertheless, however, screening of such measurements may be desirable since there may be biological systems (for example human beings) who do not want their information state to be measured. Conventional screening such as iron, lead, water, etc. is, however, not suitable since the low-energy quanta do not interact sufficiently with these materials.

According to the invention, a so-called clearing system is used for screening an entropy sink, which can interact with all the very low-energy quanta that are known. Entropy therefore does not flow from the technical installation to the instrument but into the entropy sink, which means that the system cannot be measured. In this case, the entropy in the sink must be less than the entropy in the respective instruments in order that the entropy gradient leads from the system to the clearing system, and not to the instrument.

The entropy sink is a suitable random number generator which is designed such that it can interact with the respective quanta. This is designed, for example, with regard to the wavelength of the quanta to be received. In this case, for example, the boundary layer of a semiconductor is designed such that a spatially crossing-free chain of electrons or holes is created which have the predetermined path length (depending on the wavelength of the quanta).

Random number generators are technical aids for receiving low-energy quanta. The energy as well as the information of the quantum are received during this reception process. The information can be filtered, evaluated and stored by downstream circuitry. Important problems relating to the transmission of information (messages, data) from a biological transmitter to a technical receiver are the solution a) of the addressing, that is to say the selection of the received information at the receiver B from the information mixture in the environment, and b) the interpretation of the changes to the random number generator.

Solutions to both problems will be described in the following text:

a) Addressing or selection

Addressing is carried out by transmitting addresses of the transmitter to the receiver. Addresses are, for example, a resonance key or surrogate of the transmitter. The transmitter transmits its information to the environment all the time. The problem at the receiver is to filter out this information. Since the low-energy quanta can be transmitted over a very long distance, all the possible quanta, that is to say even those from transmitters a very long distance away, are superimposed in the receiver. The receiver has to filter the quanta of the transmitter from these superimpositions.

There are a number of methods for selection. On the one hand, the method of calibration between the transmitter and receiver, see the following paragraph b), and on the other hand the identification of the transmitter on the basis of its individual transmitter features. Since the transmitter is not selected on the basis of determination of signal amplitudes, the distance between the transmitter and receiver is also of secondary importance.

In nature, quanta are exchanged all the time. This results in a change in the state of microparticles. One option for information storage is, for example, storage of information in the spins of microparticles. Since quanta are exchanged all the time, every object in nature and technology always influences its environment and is in turn influenced by its environment. This influence may be made useful by suitable selection. If, for example, an object A produces a surrogate (by cell sap extraction), then the newly created, natural object A1 (the surrogate) exchanges quanta all the time with the object A. Since A1 has been produced from A, both objects oscillate with the same energy and at the same frequency. They are, so to speak, “entangled”, for which reason they carry out specific information exchange.

Every material production process results in entanglement between the original (A) and the duplicate (A1), in the respect that the original and duplicate exchange information all the time, and the information exchange can be filtered out from the other influences from the environment. The original and duplicate have a resonance relationship, so to speak, since they transmit and receive at the same frequency.

Two alternative viewpoints are possible for the physically implemented entanglement, but both have the same technical application options.
i) The entanglement must not be understood in quantum-mechanical terms, since it is not true that what happens to the object A will also happen at the same instant to the object A1, in the sense of the known remote effect of entangled quantum states. The entanglement simply means fine tuning of the frequency such that the original and duplicate can exchange information.
ii) The entanglement must be understood to be quantum-mechanical, that is to say that which happens to the quanta of the object A also happens at the same instant to the quanta of the object A1, in the sense of the known remote effect of entangled quantum states. However, since no absolutely identical duplicate exists, then the effects of the changes in A can admittedly be received at that instant in A1 but, since A1 still also has different quanta than A, the state of A1 does not change identically to the state of A. Only the entangled quanta of A and A1 change their states identically.

Both i) and ii) can be technically made use of in the same manner by setting a receiver to the frequency of a transmitter.

There are therefore three options for addressing:
1.) The addressing of a transmitter A in the receiver B can be carried out by any type of surrogate A1, that is to say parts of the object of A itself, digital fingerprints, identical components (for example identical diodes in the transmitter and receiver), unique serial numbers etc. By way of example, the surrogates are inductively or capacitively coupled to the tuned circuit of the semiconductor component that is being used, by means of a specific device (plate capacitors, windings, measurement cups).
2.) Another option for addressing is the alignment of the receiver with the desired object with corresponding measurement probes, antenna installations or collimators.
3.) A further simple option for addressing is provided by the choice of the sampling frequency. Transmitter objects and receivers produce noise over a very broad spectrum. By the choice of its sampling rate, the receiver decides which quanta it wishes to receive, and with what energy. If, for example, the aim is to receive quanta whose energy is E=5.3*10⁻³³ J, that is to say 8 Hz quanta, because the brain frequencies of a human being are intended to be evaluated, a suitable sampling rate for the noise generator is 16 Hz. Higher-frequency noise components were produced significantly by other quanta. All of these information items are superimposed at the generator to form the typical, known noise signal of the noise generators. The crucial factor for the evaluation algorithm that is used is whether the “pure” 8 Hz values are used or whether the noise generator is nevertheless sampled at a higher rate, with only the 8 Hz mean values being included in the further processing.

By way of example, one technical option for selection is as follows:
Taking of blood or cell sap from A and production of an object A1 (the surrogate) in this way
Physical transmission of the object B to a technical receiver B
Coupling of the object A1 to the noise generator of B, for example such that the surrogate is located over a measurement cup between the capacitor plates of the capacitor, for example as part of a tuned circuit.
   It is also possible to connect the surrogate capacitor in parallel with the feed voltage of the noise generator.
   It is also possible to use a headset as a noise source, connected in a suitable manner to the noise generator.
The selection process is complete.

The surrogate therefore capacitively influences the tuned circuit and, via the entanglement of the object A1 with its original A, the random number generator filters the information from A out of the information mixture that is received all the time, even when the objects B and A are physically a long distance apart from one another.

b) Interpretation or calibration
b1) Motivation for calibration

At the moment, there are various projects throughout the world in order to identify patterns from global or local noise data, and to interpret these patterns in order to make predictions or for correlation. The so-called Global Consciousness Project at Princeton University is known⁸, in which noise generators have been installed throughout the world for 20 years and attempts have been made in this time to correlate the results of the noise measurements with global events such as earthquakes, volcanic eruptions, and terrorist attacks. ⁸ noosphere.princeton.edu

One important aim in this case is to identify whether the statistical characteristics of the noise signals change before or after global events. The aim in this case is to form an indicator or prognosis for specific global events.

These projects have been successful to a greater or lesser extent. This is because of the fact that the statistical characteristic values relating to global events have a random behavior. The main reason is that incorrect characteristic values are being searched for.

If the low-energy quanta are considered to be part of an alphabet of a communication language, which is still unknown to us, of technical and biological systems, it becomes clear that the analysis of the occurrence of mean values, median values, scatters, etc. cannot indicate any actual relationship with the events, of whatever type. If one were to statistically examine the distribution of characters in a literary work, for example a novel, one would never discover a relationship in the novel in which one could see that a specific action in the novel had already been indicated in the previous chapter. At the semantic level, the action is, of course, indicated, but a relationship such as this can never be found on the statistical levels of counting the frequencies of letters and the like (the abovementioned projects in fact do nothing more). Only if one really understands the letters and can form words from them, in turn joined together to form sentences, in order then to identify the semantics of a sentence can anyone make predictions about the further progress of the text by analysis of a text.

All of the abovementioned projects which wish to make predictions about events from statistical patterns in the time sequences of noise data will therefore fail if the predictions are intended to include a certain level of complexity and non-triviality.

One particular problem in the analysis of noise data is also that the influence on the noise processes being examined by quanta from other objects and processes (also at a long distance) could in principle all be filtered out of the noise data. The only important factor in this case is to select the respectively correct filters, then complex patterns or else simple repetitions can be found in the noise data. However, it must also be remembered here that the patterns found will sometimes themselves only be artifacts of the method, that is to say patterns which are produced only by the analysis method. Every examination must therefore be limited in time, although this means that multiplication of the noise signal by a time window and mathematical convolution of the examined random function with a square-wave function in the image area of its Fourier transforms, which in turn results in periodicities that are method-dependent. Particularly if the examinations analyze trivial relationships, that is to say correlation, histogram similarities, subordinate frequencies, fractal structures, mean-value discrepancies, drift etc., is it possible to find precisely what one was looking for in the noise data.

However, even if one excludes these method errors, the desired information can generally not be found by means of the abovementioned statistical evaluation processes since the correlations that are sought, for example between noise values from random number generators and global events, exist only in the trivial case. Nevertheless, global events in the noise sequences from random number generators can and will be indicated in advance, although they can only be found using the present-day methods of statistical and stochastic analysis of random processes.

Significant results can be achieved only by considering the noise data as an alphabet of noise values which are produced by quanta. However, according to the invention, this means a transition from the purely statistical and stochastic analysis of random processes to a semantic analysis of these sequences. This is because random sequences form letters, words and sentences of an information exchange which is physically created by quanta.

Each quantum can now store and transmit a plurality of bits of information, which means that it would be possible to transmit complex texts by the sequence of a plurality of quanta. Only the alphabet of these complex texts is unknown.

However, even if one does not know the alphabet of quantum information as postulated above (this is not known in particular in the case of natural systems), complex information can be transmitted in that both the transmitter and the receiver of the information can control a coding and decoding method, which is admittedly unknown but has nevertheless been agreed on, that is to say by both sides defining a semantic.

The possibility of a complex (and therefore semantic) information exchange between a transmitter and a receiver is provided by the calibration process. The calibration is necessary when signals from nature (for example from the biological system, human beings) are intended to be received and interpreted, since it is in fact impossible to deliberately affect the quantum emission from the transmitter.

b2) Calibration

In order to significantly improve the results of reception using random number generators, the generators must be calibrated in this context when they are intended to receive relatively complex information items. In this case, the calibration defines the semantic level between the transmitter and receiver.

A simple calibration process, that is to say tuning between the transmitter and receiver by means of the information content of the messages to be exchanged, in the example of a “calibration by means of the level of the entropy” in the transmitter can be technically integrated in the process, for example, as follows:
Addressing of transmitter A in the receiver B by use of an identifier ID, surrogate of the transmitter
Defined increase in the entropy of the transmitter (for example by heating) and transmission of entropy quanta. One specific entropy increase is, for example, batteries being drained since the maximum entropy is released during the draining process. If the batteries have been drained in accordance with a random key, the receiver can be readjusted (calibrated) until it can measure this entropy released.
Reception of the entropy quanta at the receiving noise generator, whose behavior is influenced by the quanta but is still random or statistically appears to be random
Processing of the amplitude values of the noise generator by means of a specific algorithm, and generation of a number or numerical sequence
Interpretation of the numerical sequence as high or low entropy in the transmitter, and checking whether this corresponds to the facts in the transmitter.
Calibration:
   When the statement of the receiving noise generator is correct for the user (high entropy measured, when high entropy is present), the calibration process is continued using different entropy values of the transmitter.
   When the statement of the receiving noise generator is, however, incorrect for the user, then the parameters of the noise generator and of the evaluation algorithm must be adapted systematically with the same transmitter setting (for example changing the value range of the noise generator, the sampling rate of the noise generator, the coefficients of the algorithm, normalization), to be precise until the information emitted (and known) by the transmitter has been received correctly in the receiver.
   Following this, continuation using different transmitter settings.

After the calibration, the receiver will have been set to the low-energy quanta of the transmitter and can correctly interpret subsequent quanta, that is to say the transmitter sends information on whether it has high entropy, then the calibrated receiver receives this entropy correctly in that it “randomly selects” a numerical sequence, which is identified as having high entropy in the subsequent algorithm. The semantic is defined.

However, this means that different receivers which have also been calibrated differently for various reasons may react differently to the same information from one transmitter. However, this has been known for a long time from the automata theory. This means that since a complex receiver of quanta generally has an internal state and a specific algorithm for processing the quantum information, an identical message in the receiver (an identical quantum or a sequence of quanta) can lead to different “deflections” or interpretation. Calibration of a receiver is therefore required.

If this calibration is not carried out, then a third party (uncalibrated eavesdropper) cannot so easily decode the information to be transmitted from the random numerical sequence. For such an eavesdropper, this remains a random numerical sequence without any semantic meaning. This is because different random numerical sequences may have the same semantic meaning in the calibrated receiver, and identical random numerical sequences may have a different meaning for different receivers. The desired information can therefore actually reliably be identified by the process of calibration and addressing. Data communications based on low-energy quanta therefore cannot be identified as easily by third parties without background information.

As stated above, nature uses a complex alphabet for exchanging information, whose “raw character chain” is represented by the random values from noise generators. The previous statistical evaluation of random sequences, that is to say the analysis of sequences of noise amplitudes, has only very limited success (or none at all) however. Therefore, according to the invention, it was necessary to calibrate a receiver since in this way the transmitter A and the receiver B will have agreed on the content of noise sequences, and can therefore communicate with one another.

If, for example, both the transmitter and the receiver are random number generators, then both generators can and will generate completely independent numerical sequences and, despite this, they can exchange not only energies (low-energy quanta) but also complex information items (for example “transmitter has high entropy”) by virtue of the previous calibration.

High and low entropy values may in this case be coded as “1” or “0”, thus allowing any desired data to be transmitted (as a binary numerical sequence).

Transmitters (a biological system) and receivers (noise generators with a processing unit) can communicate with one another according to the method by implementation of the addressing and calibration.

The addressing was necessary in order to set up a point-to-point link between a biological system and a receiver. Another form of data transmission for the purposes of a broadcast link such as in radio can be carried out without addressing. All that is necessary to do this is for the receiver to be set to the appropriate frequency. However, according to the invention, no actual frequency is used, but the transmitter and receiver use a so-called resonance key. In a biological system, the entropy of a biological object (for example a bacteria culture etc.) will actually not be changed in a random clock cycle (this is the resonance key) for a time interval At An increase in the entropy in the transmitter is understood semantically, for example, as 1, and no increase is understood, for example, as 0. The receiver can now check and identify the noise of its own local random number generators (avalanche diodes, transistors) in time with the random key, and can identify whether a 1 or a 0 has been produced in the biological transmitter. The entropy transport always works, but only the receiver which is sampling its own noise signal using the agreed random key can identify whether the transmitter has actually increased the entropy (semantically a 1), or not, using this key.

The method makes use of a natural characteristic of compensating for existing differences. However, differences exist not only of an energetic nature (for example temperature differences, potential differences) but also differences relating to entropy and in the end information items. The natural characteristic of the continual equalization of entropy can be utilized by means of the abovementioned method.

It is also possible to exchange information differences directly, although information is not an absolute parameter but is always a relative parameter with respect to a previously selected semantic level. Only when the receiver is at the same semantic level as the transmitter can it identify information as such at all, and go into resonance with it. In order to understand the noise signal which a human being emits, it must have been calibrated in advance, in order to produce the semantic level.

Noise generators produce noise over a very broad spectrum. By the choice of its sampling rate in the A/D converter, the receiver decides which quanta it wishes to receive, with what energy. For example, if the aim is to receive quanta of energy E=5.3*10⁻³³ J, that is to say 8 Hz quanta, for example because brain frequencies have to be evaluated, a noise generator sampling rate of 16 Hz is suitable. Higher-frequency noise components were essentially produced by other quanta.

From time to time, one reads in the literature of white noise being used as a carrier of a novel communication channel, which has not yet been discovered. However, the white noise is not the carrier of information modulated onto it, but the white noise is the information itself. This is because low-energy quanta have the physical characteristic of extending spatially very widely and of expanding, for which reason a novel information technology need not modulate any information onto a carrier wave. The information of a transmitter is already received at the speed of light (or possibly higher) at the receiver, and all that is necessary is that this must in fact be able to receive this.

The information of a transmitter object is transmitted by existing natural transmission mechanisms, a large spatial and time extent of quanta, and their major penetration to the receiver.

The novel data communication described here easily reads the information sent all the time by each object from the noise. According to the invention, nature carries out the actual data transmission itself, so to speak. The major content of the invention is therefore, based on novel receivers, to use random number generators to receive the low-energy quanta carrying information, and then selectively to filter them out. Specific addressing and calibration are required for this purpose.

The method can in principle be carried out in any frequency range. The technical advantage of low-energy quanta is that nature provides data transmission itself, so to speak, since one is located in the near area of the biological transmitter, and the longitudinal components of the wave can therefore be used for transmission. It is therefore irrelevant for the invention whether one considers the quanta with a large spatial extent in the order of magnitude of their wavelength around the biological system (novel aspect of this description) or whether one makes use of the longitudinal characteristics of the near area of electromagnetic waves. The technically resultant effects are equivalent.

One major component of the invention is to not only replace old known methods from information technology by cheaper or more efficient methods, but to use the invention to create completely novel application options. For example, this results in completely new capabilities for remote diagnosis of patients, therapy capabilities or communication with the very seriously disabled.

Inter alia, the invention makes possible the following technical applications:
1. Reception, evaluation, storage of information from biological and natural systems, such as human beings, animals, plants, minerals and materials for obtaining information
   Applications in the case of human beings, for example
      Diagnosis of illnesses
         In contrast to known methods and facilities (for example devices in the Oberon class (Clinictech Inc.⁹, Austin, Tex., USA; IPP, Omsk, Russia), Quantec (USA), entropy quanta of the organs are received directly by a random number generator (RNG) and are processed further in a PC (PRZ) Neither trigger sensors, measurement plates, headsets or other aids are required for this purpose. 9 Clinictech Inc., 10509 LaCosata Drive, Austin, Tex. 78747, USA
         The method described here therefore allows remote diagnosis of biological systems, since the entropy quanta (LEQ) can also be transmitted and received over very long distances.
         The expressions used in a confusing manner by other appliances and other developers, such as quantum information, entropy quanta etc. have nothing to do with the low-energy quanta expressions introduced in the invention (entropy quanta) since the abovementioned appliances (Oberon, Quantec, etc.) are based on unknown scalar waves, radionic fields, probability waves, ghost waves.
         The low-energy quanta described in this invention are, however, the classical field quanta (for example photons) with the characteristic of extremely low energy (frequency), thus overall resulting in the effects explained here. There is therefore no need to postulate any new field theory, energetic theory, oscillation medicine, etc. for the applications described in the invention.
      Diagnosis of mental states
         Since the brain has a specific entropy content for specific mental states, and emits this via entropy quanta, the mental state can be diagnosed.
      Communication with the very severely disabled
         The very severely disabled can learn to initiate specific actions on a PC by means of training and calibration of the noise generators, thus making it possible for them to initiate actions on a PC on their own, as they require.
      Diagnosis of settings relating to specific situations
         Since states can be diagnosed, the setting relating to question contents can also be determined, by suitable questioning.
      Determination of the truth of statements
         Since mental states can be diagnosed, the truth of the response can also be determined by suitable questioning.
   Applications in the case of animals, for example
      Diagnosis of illnesses
      Diagnosis of mental states
      Communication with animals
         Since mental states can be diagnosed, the animals feelings during specific actions can be determined.
      Knowledge acquisition about animals
   Applications in the case of plants, for example
      Diagnosis of illnesses
      Knowledge acquisition about plants
      Medicament manufacture
         It is possible to determine whether medicaments are compatible with a specific person or a group of people.
      Compatibility analysis of plants for human beings
         It is possible to determine whether plants are compatible with a specific person or a group of people.
   Applications in the case of minerals, for example
      Diagnosis of illnesses
      Knowledge acquisition about minerals
      Medicament manufacture
      Compatibility analysis of minerals for human beings
   Applications in the case of natural materials, for example
      Knowledge acquisition about materials
      Medicament manufacture
      Compatibility analysis of materials for human beings
      Exploration of natural resources or other materials under the ground, under water or in other locations
2. Reception, evaluation, storage of information from biological and natural systems a (long) distance away physically, such as human beings, animals, plants, minerals and materials for obtaining information
   All applications in paragraph 1), in which case the natural systems to be measured may be a long distance physically away from the receiver (up to considerably more than a thousand kilometers).

Three technical applications of the invention will be mentioned by way of example in the following text.

1.) It is possible by means of the method according to the invention to objectively read information states of a biological system by constructing information sinks which enter resonance with specific desired information in the transmitter. In the same way that one can objectively diagnose mental states of people, since the states correspond to certain entropy relationships which can be received by receivers that are suitable for this purpose, other brain states of a person or of a biological system can also be detected by measurement. In contrast to conventional methods using EEG signal evaluation, which is incorrectly carried out in the high-energy area (in the view of the invention), low-energy quanta, which represent specific brain states of a person, can be received and evaluated by the reception of quanta by means of noise generators.

Applications relating to this are diagnosis systems, lie detectors, communication systems for the very severely disabled, and therapy appliances.

2.) The method according to the invention can also be used to read illness states of a human being objectively at relatively long distances, by constructing receivers which receive quanta which correspond to the energy of the transmission to be expected.

The advantage of the method is that this also makes it possible to monitor the very severely ill, which is not possible for a doctor or hospital.

3.) It is known that there are certain groups of people who can use various instruments, such as pendulums or divining rods for example underground water flows or raw material resources, and to carry out other activities. These activities are nowadays not considered to be serious since they often cannot be checked or at least are not reproducible.

With the low-energy quanta receivers described here, all of these so-called radionic activities can be constructed and implemented reproducibly by technical appliances, and this will be explained using the example of a single-handed divining rod.

It is known that someone carrying a divining rod must calibrate this in advance since, in fact, it is actually not known what non-voluntary muscle deflections lead to the respective reactions of the divining rod in what question situations. After calibration, the divining rod can respond correctly to relevant question situations for the user, since the muscle movements are in fact produced involuntarily, and the divining rod only produces a response such as this with the intention of creating an involuntary action by a person which could not penetrate to the consciousness because of different nerve activity in the brain.

These tasks of specially trained personnel can be carried out technically by so-called “electronic pendulums” (ELPs).

By way of example, an ELP operates as follows: a thermal noise generator, for example a zener diode, is used as a noise source, as the specific receiver of low-energy quanta. This analog noise source is then sampled, for example at a frequency of 15 Hz, and is digitized. The binary random number sequence that is produced is then evaluated in a PC for a predetermined time interval, for example of 5 seconds.

An ELP must be calibrated on the basis of its technical implementation. In this case, a first question is chosen from a set of about 100 questions (whose correct responses are all known), and this first question is then preset for the ELP. The check of the ELP is then started, and the response is awaited. During the check, the number of zeroes and ones—which the noise source has produced—is counted and evaluated over a time interval. If, for example, more ones were to occur than zeroes, then this can be interpreted as “yes”, and vice versa. If one agrees with the response, the next question is then selected, and the calibration procedure is repeated. If the response is not correct, the algorithm is adapted (for example changing the value range, changing the processing algorithm for noise data). The calibration of the ELP is carried out until the ELP has responded to about 85% of the questions in the manner expected by the user. The ELP can then be operated in the user mode and responds correctly to newly asked questions on a more than statistically expected level.

The correctness of the responses is higher than the statistical expected value because the “operator and ELP” system has learnt to give correct responses during the calibration. The learning process is carried out in such a way that the low-energy quanta emitted by the human being influence the random number generator of the ELP, in the example the thermal noise generator, in such a way that the exact random value which represents the correct response is actually produced. The calibration is therefore necessary because 1) every person emits quanta of somewhat different energy (and) information and 2) the “operator and ELP” system must also be set to the specifically implemented algorithm for evaluation of the numbers.

All random number generators of suitable design can be used as a noise source for ELPs. However, in practice, the body noise of the operator himself, for example, can also be used as a noise source. So-called otoacoustic noise signals (that is to say noise generators which can measure and process the noise in the inner ear) or systems for measurement of fluctuations in the skin conductivity as a noise source, can be used for this purpose. The ELP can also be worn as a type of clock with a metallic base directly on the skin on the arm, and can be used in a mobile form. Further mobile options would be implementations in a mobile telephone, in an organizer etc. Providing that it has previously been correctly calibrated, the ELP can therefore, so to speak, provide the answers which the subconsciousness of the person would have wished to give to the question asked.

ELP systems can also be used for other purposes such as knowledge generators, lie detectors or for medical therapy in order to provide a reminder of things which have left the consciousness.

One specific technical application example of the method according to the invention is illustrated in FIG. 2.

The biological system comprises a bacteria culture of e-coli bacteria (BIO), which have been grown in a number of Petri dishes, a device for pipetting of toxin (DEV), in the exemplary embodiment high-percentage alcohol, and drive electronics for initiation of the pipetting process (RNGA). The overall system of the bacteria including pipetting is referred to as the transmitter (A). The receiver comprises an avalanche diode (DIO) within a tuned circuit for production of a noise signal, an operational amplifier (OPV), a A/D converter (AD) for conversion of the noise signal to a digital signal (BITS) and a processing unit (laptop, not illustrated). The transmitter and receiver are screened, battery-powered and are at a distance of about 10 m from one another.

At the transmitter end (A), a random number generator is used to decide whether the toxin—which is directed at an inserted Petri dish with e-coli bacteria—will be supplied at a frequency of 1 Hz in the following time interval (for example Δt=1 minute) in order to increase the entropy by the process of killing the bacteria culture (which is semantically coded as 1) or whether no toxin should be administered. Once this time interval has elapsed, the random number generator (RNGA) once again decides whether the process should be repeated for the next inserted Petri dish, or whether the toxin should remain switched off for the next time interval (Δt=1 minute) (which semantically means a 0).

An avalanche diode (DIO) is used at the receiver end (B). The diode noise at the receiver end is amplified by an operational amplifier (OPV), is sampled at least 2 Hz (AD), is digitized, and is transmitted to a receiver computer as a digitized noise signal (BITS). The receiver computer evaluates the noise for example by forming the distribution functions (amplitude density function, that is to say histograms) of the respective time periods Δt. The receiver uses the change in the distribution function of each time interval to identify whether the entropy of the bacteria culture has been increased by the toxin at the transmitter end (semantically a 1), or not (semantically a 0).

In this case, it should be stressed that the avalanche diode in the receiver changes its noise signal characteristics (amplitude density function) in time with the entropy increase of the bacteria culture at the transmitter end, even though both the transmitter and the receiver are completely screened according to the normal method for communication technology and are also not connected via the electrical power supply. The bacteria culture transmits a change in its entropy all the time to its environment and thus influences all the objects in its environment, which therefore enter resonance, for example the avalanche diode in the receiver, even when this is a long distance away. At the receiver end, the signal characteristics (amplitude density functions) apparently change randomly, but it is possible to identify by matching with the transmitter information that their signal characteristics are changing precisely in the random rhythm of the entropy increase.

The actual signal transmission is provided by the natural process of entropy equalization between the bacteria culture (transmitter) and receiver which, by virtue of its characteristics, takes place over long distances. According to the invention, technically usable signal transmission is achieved by suitable reading in the receiver, making it possible to identify whether the entropy of a biological system has or has not increased.

The entropy transport method disclosed above from a biological system to a technical receiver is generally applicable. Building on this embodiment variant, it is therefore possible to produce a system which can identify the entropy state of relatively complex biological systems, and can also measure the entropy state of individual organs. By way of example, this makes it possible to produce a diagnosis system which determines the entropy state of human beings and their organs. By means of the calibration process with organs which are already ill, the algorithm can be adjusted at the receiver end such that, however, it corresponds only to a certain organ change (entropy change). The method can also be used to measure the entropy content of a brain, in order in this case to gain knowledge about internal processes in the brain.

The addressing of the biological object and of individual organs is carried out as described by means of biological surrogates, by means of a surrogate cup and capacitive coupling of the surrogate to the noise generator in the receiver.

A simpler variant of the addressing process is implemented when the frequencies of the individual biological subsystems are known. In this case, the addressing is carried out by choice of the sampling frequency in the A/D converter in the receiver.

### FIGURE DESCRIPTIONS

FIG. 1

BIO Biological Environment

EQ Energy Quanta

LEQ Low-Energy Quanta

s Distance

RNG Random Number Generator

PRZ Processor

DEVICE Device

FIG. 2

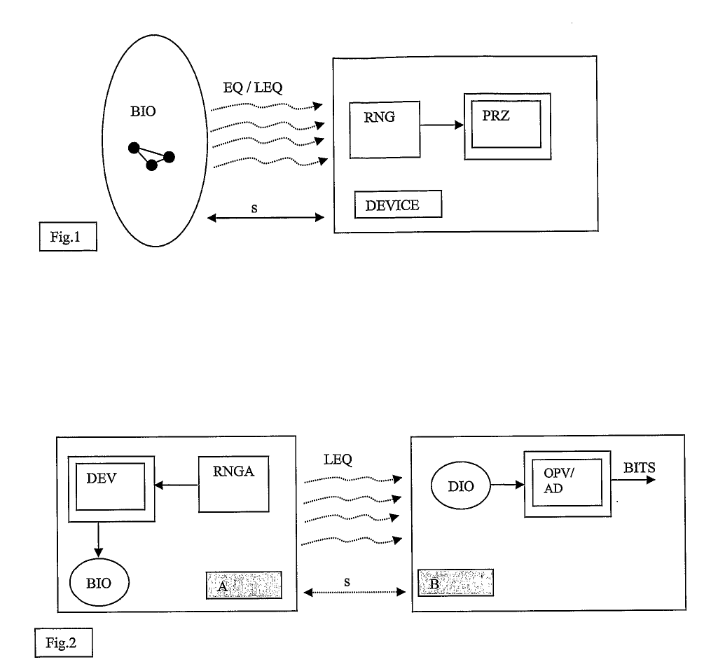

BIO Biological Environment

DEV Device

LEQ Low-Energy Quanta

DIO Diode

RNGA Random Number Generator A

BITS Bits

OPV/AD Operational Amplifier

1-15. (canceled)

16. A method for measuring information of biological systems, said method comprising steps of
providing suitable receivers formed as noise generators for receiving and evaluating low-energy quanta LEQ with a frequency in the range between 1 Hz and 100 Hz or very low-energy quanta LSTEQ with a frequency of less than 1 Hz,
receiving said low-energy quanta or very low-energy quanta by said receivers,
evaluating said received low-energy quanta or very low-energy quanta with the physical relationship between frequency and energy being used in order to determine the energy of the low-energy quanta or very low-energy quanta to be received and in order to use the noise generators as receivers or transmitters of low-energy quanta or very low-energy quanta,
time sampling the noise signal generated when receiving low energy quanta or very low energy quanta,
selectively filtering out the received low energy quanta or very low energy quanta from the noise signal.

17. (canceled)

18. The method as claimed in claim 16, wherein the received quanta originate from human beings.

19. The method as claimed in claim 16, wherein the received quanta originate from natural systems such as animals, plants, minerals or other materials.

20. The method as claimed in claim 18, wherein the receivers, which are based on noise generators, of low-energy quanta are used for diagnosis of illnesses, or for diagnosis of mental states.

21. The method as claimed in claim 18, wherein the receivers, which are based on noise generators, of low-energy quanta are used for communication with the very seriously ill.

22. The method as claimed in claim 18, wherein the receivers, which are based on noise generators, of low-energy quanta are used to determine the truth of human statements.

23. The method as claimed in claim 16, wherein the received quanta originate from systems which are physically a long distance away, thus making it possible to carry out remote diagnoses of biological systems or remote monitoring of technical systems and installations.

24. The method as claimed in claim 16, wherein the receivers, which are based on noise generators, of low-energy quanta are used for exploration of natural resources.

25. The method as claimed in claim 16, wherein the receivers, which are based on noise generators, of low-energy quanta are used for determination of materials, and these materials can thus be located specifically by calibration of the receivers for the corresponding materials, which makes it possible to select those quanta which those materials permanently emit, from the range of signals.

26. The method as claimed in claim 16, wherein the receivers, which are based on noise generators, of low-energy quanta are used for data communication, in that addressing and calibration are carried out between the transmitters and receivers of quanta, as a result of which the receiver can filter out the quanta sent via the transmitter from the information mixture of its noise generator, and can thus transmit a bit sequence from the transmitter to the receiver.

27. The method as claimed in claim 16, wherein the method is carried out in the following steps:
addressing of transmitter in the receiver by use of an identifier, surrogate of the transmitter
defined increase in the entropy of the transmitter and transmission of entropy quanta
reception of the entropy quanta at the receiving noise generator RNGB, whose behavior is influenced by the quanta but is still random or statistically appears to be random
processing of the amplitude values of the noise generator by means of a specific algorithm, and generation of a number or numerical sequence
interpretation of the numerical sequence as high or low entropy in the transmitter, and checking whether this corresponds to the facts in the transmitter calibration.

28. The method as claimed in claim 27, wherein the calibration has the following steps:
when the statement of the receiving noise generator is correct for the user, the calibration process is continued using different entropy values of the transmitter
when the statement of the receiving noise generator is, however, incorrect for the user, then the parameters of the noise generator and of the evaluation algorithm must be adapted systematically with the same transmitter setting, to be precise until the information emitted (and known) by the transmitter has been received correctly in the receiver
following this, continuation using different transmitter settings.

29. The method as claimed in claim 16, wherein the receivers, which are based on noise generators, of low-energy quanta are used for setting up and for application of computer-aided divining systems (ELPs), in that a calibration process matches an ELP and its user to one another, as a result of which the ELP responds correctly more than statistically expected when subsequently checked.

30. A device for measuring information of biological systems, said device comprising
a transmitter for generating signals or quanta and
a receiver for receiving these signals with a noise generator, wherein
the transmitter has a biological material, a device directed thereto for pipetting and a control for resolution of the pipetting and
the receiver contains a diode which is connected to an operational amplifier.
